(19) United States
(12) Patent Application Publication
(10) Pub. No.: US 2020/0326346 A1
(43) Pub. Date: Oct. 15, 2020

(54) LUMINESCENT RESONANCE ENERGY TRANSFER SENSORS FOR NON-INVASELY AND CONTINUOUSLY MONITORING GLUCOSE FOR DIABETES

(71) Applicant: JIN ZHANG, London (CA)

(72) Inventors: Jin Zhang, LONDON (CA); Xianbin Wang, London (CA)

(21) Appl. No.: 16/712,245

(22) Filed: Dec. 12, 2019

Related U.S. Application Data
(63) Continuation of application No. 14/867,910, filed on Jan. 13, 2016, now abandoned.
(60) Provisional application No. 62/056,195, filed on Sep. 26, 2014.

(51) Int. Cl. G01N 33/66

(52) U.S. Cl. CPC G01N 33/66

(57) ABSTRACT

The present disclosure discloses a non-invasive wireless glucose level monitoring device. The device is fitted into a contact lens and can analyze glucose levels in tear fluid. The results of the analysis can be continuously transmitted to a nearby receiver and uploaded to a computer, mobile device or server.

### FIELD

The present disclosure is related to an encoded wireless sensor system for diabetes with a manner of continuous, non-invasive measure.

### BACKGROUND

Diabetes mellitus, resulting from the disability of pancreas to secrete insulin, is quite prevalent to children and young patients. Without regular insulin treatment to keep the normal glucose level in blood, it can cause kidney failure, heart disease, gangrene, blindness, and even death. Artificial Pancreas (AP) is regarded as “the most revolutionary development in diabetes care” (1), which has been constructing a closed-loop system to automatically deliver insulin to realize the efficient treatment of type-1 diabetes even at home. The results of the AP Project conclude that a continuous glucose monitor/sensor (CGM) is vital to ensure proper insulin delivery, and to avoid the hyperglycemia.

Compared to the conventional glucose blood test, e.g. finger-prick, the CGM sensors do not provide a “snapshot” picture for patients, but the overall trend within along period to help patients with insulin delivery for efficient control their blood glucose level. Most recent development of CGM is related to invasive (implant) sensors for glucose monitoring in the interstitial fluid (2-5). However, the serious concern is related to the bio-instability of the implanted CGM due to the subcutaneous inflammatory reaction (6). Although recent research studies show that biocompatible coatings can improve the tissue biocompatibility of the implant devices, it may take years to eventually include the implantable CGM in the artificial pancreas for efficient treatment of type-1 diabetes. Moreover, the surgery for inserting or removing the sensor from subcutaneous tissue is painful to patients, and may bring extra burden to the health care system. Thus, a non-invasive and continuous glucose sensor is now in high demand as an alternative CGM for the artificial pancreas. Meanwhile, an efficient tracking network by applying most recent wireless technologies will help doctors/caregivers and patients work together to improve life quality of patients.

Tear fluid is to clean and lubricate the eye, and nourish the cornea. It has been demonstrated that there are over 20 components in tears, including salt water, proteins, glucose, and some small metallic ions, etc. (7). Diagnosis of bimolecular in tear fluid, such as ocular rosacea, has been performed primarily to clinicians for the high molecular-mass glycoproteins in tears (8). The detection of ocular glucose dates back to 1930 (9). Following that, Michail and his collaborators first demonstrated that the level of glucose in tears is often increased in diabetic patients (12, 10). Sen and Sarin studied over 200 cases, their statistic results showed that the blood glucose is about 2 times higher in diabetic patients compared to that in non-diabetics, whereas tear glucose levels are 5 times higher in diabetics than that in the general population (11). Moreover, most recent studies indicate that tear glucose mean values were 0.35±0.04 mmol/L and 0.16±0.03 mmol/L, respectively, for diabetics and healthy ones (12). Moreover, Gasset et al., and Motoji. et al., found a definite relationship of tear glucose and blood glucose and concluded that hyperglycemia could be detected by measuring tear glucose level (13, 14). Dawn and Hill reported the correlation coefficient ( ) between blood and tear glucose levels was +0.53 (15) in healthy ones.

Decades of research on tear glucose demonstrate that the tear fluid can be used for the glucose level diagnosis (16). However, it is particularly challenging in measuring constituents from tears. Firstly, it is difficult to acquire enough tear samples in a short period of time. Unlike blood test, a blunt-end glass capillary is usually required to collect tear samples. It normally takes more than 10 minutes to collect 10 μl of tear sample required for testing (17). Secondly, the concentration of the glucose in tears is much lower than that in blood (18). Recently, Hydrogel-based soft contact lenses have been approved as a safe daily wear lenses for diabetic patients (19, 20). Indeed, contact lenses have many applications beyond vision correction. They are being considered as an alternative tool to continuously monitor the level of glucose in tears non-invasively (21-23).

It is noted that time lag in measuring tear glucose is as common as other CGM for it takes 5-15 minutes to allow the change of glucose in blood to eventually reflect in tear/interstitial fluids (24); while it is not difficult to overcome by serials of calibrations (25). The challenge of tear glucose testing is the development of a very sensitive device required to analyze the glucose level in very small amount of tear sample.

One of the present inventors (Jin Zhang) has been working on the enzyme-based nanostructured sensor incorporating into contact lens-like materials to detect the glucose in aqueous continuously and quantitatively (26, 27). With further development of the glucose sensor for detecting tear glucose, new and advanced fluorescent pair label the protein, e.g. Concanavalin A (ConA), has shown stronger sensitivity to detect glucose in a range from 0.01 mmol/L to 10 mmol/L.

### SUMMARY

The present disclosure provides an apparatus for the detection of glucose levels in body fluids which comprises a transparent substrate, a luminescent resonance energy transfer (LRET) optical sensor embedded in the transparent substrate capable of generating electromagnetic radiation in response to interaction with glucose contained in a body fluid, and a signal detector located within a detection range of the luminescent resonance energy transfer optical sensor.

In an embodiment the luminescent resonance energy transfer optical sensor is a nanostructured LRET pair-conjugated enzyme configured.

In an embodiment this LRET pair-conjugated enzyme includes a light emitting donor, a light absorbing and emitting acceptor, an enzyme coupled to the acceptor, linker molecule linking the light emitting donor to the enzyme, and wherein the interaction with glucose includes the linker molecule being replaced by glucose.

In another embodiment this nanostructured LRET pair-conjugated enzyme includes a light emitting donor, a light absorbing and emitting acceptor, an enzyme coupled to the light emitting donor and linked to the light absorbing and emitting acceptor by a linker molecule, and wherein the interaction with glucose includes the linker molecule being replaced by glucose.

A further understanding of the functional and advantageous aspects of the present disclosure can be realized by reference to the following detailed description and drawings.

### BRIEF DESCRIPTION OF THE DRAWINGS

Embodiments disclosed herein will be more fully understood from the following detailed description thereof taken in connection with the accompanying drawings, which form a part of this application, and in which:

FIG. 1 is an illustration of and embodiment of an encoded lens sensor.

FIG. 2 is a graph showing relative photoluminescence of magnetic element doped up-conversion nanostructures at various wavelengths.

FIG. 3(A) is a Fourier Transform Infrared reflectance (FTIR) of the polyethylenimine or polyaziridine (PEI) modified NaGdF₄:Yb:Er:Fe.

FIG. 3(B) is a Transmission Electron Microscopy (TEM) micrograph of the magnetic element-doped up-conversion nanostructures(Fe nanoclusters doped NaGdF₄:Yb:Er.

FIG. 4(A) shows a BRET sensor made of quantum dots used as an acceptor in the LRET sensor; and glucose sensitive protein-conjugated Renilla luciferase (RLuc) used as a donor in the sensor.

FIG. 4(B) is a scheme of glucose binding protein (GBP) linked Renilla luciferase (GBP-Rluc)recombinant protein sequence used in BRET sensor.

FIG. 5 is the spectra of the BRET sensor corresponding to aqueous media with and without glucose.

FIG. 6a is an illustration of a LRET transducer made of hybrid nanostructures coated on a silicone hydrogel substrate.

FIG. 6b is an Illustration of a patterned nanostructured LRET transducer on a silicone hydrogel substrate used for identifying the specific species, i.e., glucose in certain body fluid, e.g. tears, saliva, urine, etc.

FIG. 7 is an image of patterned optical nanostructures on hydrogel.

FIG. 8 is an Illustration of the proposed readout system by combining the charge-coupled device (CCD) optical/fluorescence detector, the Bluetooth device, and computer/smart phone.

FIG. 9 is the images of patterned optical nanostructures with reference or control areas on hydrogel showing glucose concentration dependence.

FIG. 10 is an image of the lens sensor made of three major components (1) hydrogel substrate (silicone, Poly(2-hydroxyethyl methacrylate) (pHEMA), etc.) (b) Nanostructures patterned LRET transducer (c) a hydrophilic coating which can be deposited by chemical and matrix-assisted pulsed laser evaporation (MAPLE) methods).

FIG. 11 shows BSA adsorption of silicone and its nanocomposite with/without PEG deposition by MAPLE. *Significant difference was found between 1 and 3 (p<0.05). 1—silicone; 2—silicone-SNPs; 3—Polyethylene glycol (PEG) deposited on silicone; 4—PEG deposited on silicone-solid nanoparticles (SNPs).

FIG. 12 shows the results of a cell viability study showing cytotoxicity of glucose sensor on the human osteosarcoma U2OS cell derived UTA-6 cells.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, “hydrogels” refer to materials that are formed by crosslinking polymer chains, through physical, ionic or covalent interactions and are known for their ability to absorb water. An example of a physical interaction that can give rise to a hydrogel is by thermal treatment of the liquid hydrogel precursor which, prior to being subjected to a freeze thaw cycle is a liquid or near liquid. The process of freezing the liquid precursor acts to freeze the water contained in the polymer/water mixture and ice particles causes the polymer strands to be topologically restricted in molecular motion by other chains thus giving rise to the “entanglement’ cross linking to produce the hydrogel. As used herein, the phrase “up-conversion” means a process that output photon energy is weaker than input photon energy, which reflects the emission of light at shorter wavelength than the excitation wavelength (28).

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions.

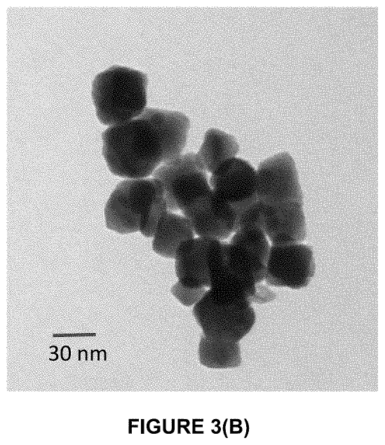

Broadly speaking, as used herein, the phrase “luminescent resonance energy transfer optical sensor”, or “(LRET)”, may refer to a sensor made of a nanostructured LRET pair-conjugated enzyme. More particularly, with reference to FIG. 4, the LRET sensor includes a light absorbing and light emitting acceptor, a light emitting donor, and an enzyme. In one embodiment, the light absorbing and light emitting acceptor is chemically bound to the enzyme, and the light emitting donor is bound to the enzyme by a linker molecule (L), so that when in contact with a fluid that contains glucose, the glucose replaces the linker molecule causing the light emitting donor to be released from the enzyme. In another embodiment, the light absorbing and emitting acceptor is chemically bound to the enzyme by the same linker molecule (L) noted above, and the light emitting donor is chemically bound to the enzyme, and when in the presence of a fluid containing glucose, the linker (L) is replaced by the glucose thus releasing the enzyme and light emitting donor. Both of these embodiments are referred to as a “nanostructured LRET pair-conjugated enzyme”.

The basis of the present sensor, is that in the presence of glucose, the light emitting donor is uncoupled from either from the enzyme (first embodiment above) or the light emitting donor and the enzyme are uncoupled from the light absorbing and emitting acceptor (second embodiment above) such that the LRET structure no longer exists in either embodiment. As will be discussed hereinafter, the consequence of this decoupling is that the light emitted from the donor is no longer absorbed by the light absorbing acceptor, and thus the light which was emitted by the light absorbing and emitting acceptor (in response to absorbing the light from the donor), changes, which corresponds to the amount of glucose present. The light emitting donor may be a fluorescent material or a bioluminescent material which both constantly emitting light.

To further develop the non-invasive and continuous glucose sensor, embodiments disclosed herein provide a luminescent nanostructured optical glucose sensor integrated into a wireless system for continuously detecting physiological glucose in body fluid other than blood, including tear, urine, sweat and saliva. The developed nanostructured luminescent resonance energy transfer (LRET) sensor can be coated on biocompatible hydrogel materials with designed patterns for improved measurement accuracy. The readout scheme can detect the changes of fluorescent properties of the glucose sensor and to send information wirelessly to appropriate one(s) who patients trust, such as family doctors and parents, to manage the disease together.

The present disclosure discloses an apparatus for the detection of glucose levels in tears, comprising a contact lens; aluminescent resonance energy transfer optical sensor embedded in the contact lens capable of generating electromagnetic radiation in response to glucose interactions; and a signal detector located within a detection range of the luminescent resonance energy transfer optical sensor.

In an embodiment the luminescent resonance energy transfer optical sensor is a nanostructured Luminescent Resonance Energy Transfer (LRET) sensor made of nanostructured Resonance Energy Transfer (RET) pair-conjugated enzyme. Additionally, the pair-conjugated enzyme can have a strong affinity to glucose. Examples of the pair-conjugated enzymes include glucose binding protein (GBP), Concanavalin A (Con A), or a combination thereof.

The luminescent resonance energy transfer optical sensor is a NIR/IR excited LRET sensor in which a donor can be made of magnetic element-doped upconversion nanomaterials. The luminescent resonance energy transfer optical sensor can also be a bioluminescent resonance energy transfer (BRET) sensor in which a donor is a bioluminescent protein.

An acceptor of the luminescent resonance energy transfer optical sensor can made of one or more materials selected from the list comprising: porous fluorescent silica nanoparticles, quantum dots, silicon, ZnO nanoparticles, nanorods, metallic nanoparticles, and fluorescent molecules.

The emitted electromagnetic radiation is a fluorescent emission in the range of visible-near infrared wavelength. The signal detector is a camera capable or detective emissions in the range of the emitted electromagnetic radiation.

Further disclosed is a process for producing Fe-doped NaGdF₄ based up-conversion nanostructures comprising preparing a first solution comprising Gadolinium(III) nitrate hexahydrate, Erbium(III) nitrate pentahydrate, Ybterbium(III) nitrate pentahydrate, Iron(III) nitrate nonahydrate and PEI; mixing the first solution into Ethylene Glycol and dispersing the solution by stirring at room temperature; preparing a second solution comprising sodium fluoride; mixing and sonicating the second solution into Ethylene Glycol; adding the second solution to the first solution dropwise at a temperature of 200° C. to form a third solution; refluxing the third solution for 6 hours; washing, purifying, centrifuging and drying the third solution at a temperature of 60° C.

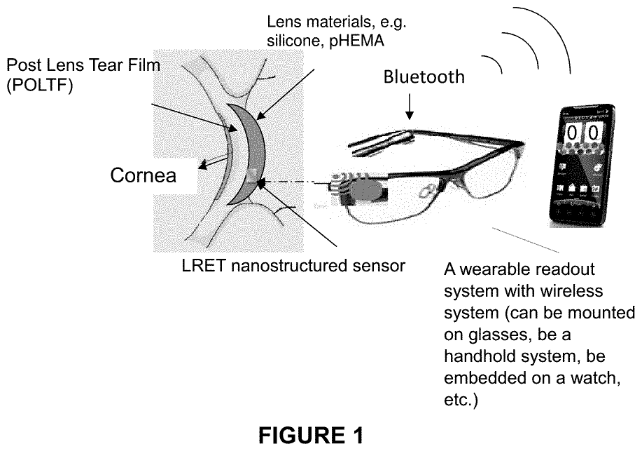

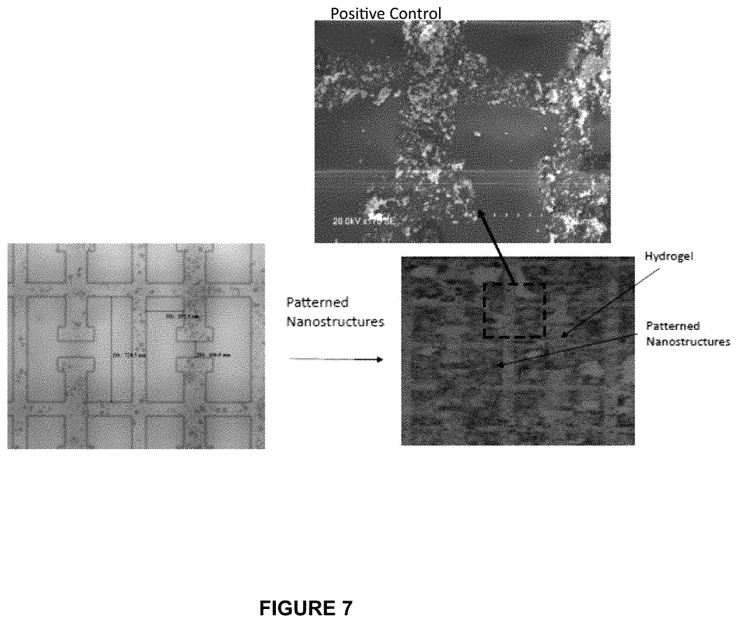

An embodiment disclosed herein is a non-invasive glucose sensor that can continuously measure tear glucose level and wirelessly send the information for efficient managing diabetes. FIG. 1 illustrates the encoded lens sensor structure disclosed herein: including (1) encoded optical glucose sensor made of a patterned nanostructures embedded in lens materials, which can be worn on the eyes, like contact lenses; (2) miniaturized optical signal detector for processing the LRET signals, and (3) optical detector connecting a bluetooth transmitter attached to a glasses, watch, or other wearable, handhold devices, which is able to communicate with a smartphone, or a computer for real-time and continuous glucose level monitoring, see system shown in FIGS. 1 and 8 showing a computer processor.

As shown in FIGS. 1 and 8, a “detector” is a fluorescence microscope/camera which can take fluorescence images of the LRET sensors and provide the fluorescence spectral responses accordingly. When the designed LRET sensor interacts with the body fluid which contains glucose, the “detector” scans on the LRET sensor, and will exhibit the different fluorescence images and fluorescence spectra of the LRET sensor depending on the amount of glucose.

In FIG. 8, the patterned LRET sensors interacting with glucose are highlighted by a rectangle (X, Y), where X and Y are the length and width of the rectangle to confine the sensing area. The negative control is highlighted by a rectangle with X′ the length and Y′ the width. The positive control are highlighted by a rectangle with X″ the length, Y″ the width. The fluorescence intensity (I) and wavelength (λem) of the acceptor and donor of the LRET sensor depending on the concentration of glucose are scanned by the fluorescence spectra. The relative fluorescence properties, e.g. intensity (1), wavelength (λem) are recoded in comparison with the fluorescence spectra of the control areas through an algebra method. On the other hand, the recorded fluorescence images taken by the fluorescence microscope/camera can be converted to the value of pixel intensity through Matlab's imaging process. The method is described as follows. First, images of the LRET sensor corresponding to the different concentrations of aqueous glucose were taken by a fluorescence microscopy/camera (40×, pixels 640×480). The external environment was kept the same during the measures. The pixel intensity and color of the recorded pixels on the images only depend on the concentration of glucose. Images are then loaded into Matlab's signal processing software by using the imread function with red, green, blue (RGB) matrix. The image file was input into an m-n−3 data array that defines RGB color components for each individual pixel. Next, the image was converted into the im2double function. In this imaging conversion process, the image matrix of the control area were used to compare with that of sensing area of the LRET sensor to obtain the value of pixels intensity corresponding to the concentration of glucose.

LRET transducer is composed of a donor and an acceptor and a glucose-affinity protein. This new luminescent resonance energy transfer (LRET) optical sensor is able to monitor glucose level for at least 5 days. It is noted that the matrix of the LRET sensor is similar to the weekly wearing contact lenses. Both fluorescence intensity (I) and resonance energy transfer (RET) as the function of time and the concentration of glucose (0.01 mmol/L˜10 mmol/L) can be measured through a readout system or a designed fluorospectrometer.

Diabetes mellitus, resulting from the disability of pancreas to secrete insulin, is quite prevalent to children and young patients. Without regular insulin treatment to keep the normal glucose level in blood, it can cause kidney failure, heart disease, gangrene, and blindness, even death. Continuous glucose monitor (CGM) is the most essential to realize a successful artificial pancreas and regular insulin treatment. Current CGMs are invasive, which may cause tissue inflammation and bio-instability of the sensor. For these reasons, the development of a non-invasive and continuous device for glucose monitoring is needed. It has taken several decades to verify that there is a correlation between glucose in tears with that in blood; however, there are several challenges to measuring constituents from tears. For instance, it is difficult to collect enough tear sample to test. Glass capillaries, normally used to collect tear samples, can take more than 10 minutes to collect 10 μl of tear sample required for testing. In addition, High sensitive glucose sensor is highly required for the concentration of the glucose in tears is much lower than that in blood.

The present disclosure provides an embodiment of a system for monitoring tear glucose with luminescent resonance energy transfer sensor by using nanostructured transducer incorporated with biopolymer lens materials for monitoring glucose non-invasively. The optical nanocomposites are transparent and highly porous nanostructures. The advantages of the nanostructured transducer include: (1) it's ability to bind to the desired bioassay for conjugating the glucose in tears; (2) the patterned coating and nanostructures enable the detection device to act as an analyte reservoir, which helps to achieve the high loading of analyte for target sensing (e.g. glucose sensing); (3) that the nanostructured sensors coated on contact lens will not interfere with patient vision, but enhance the oxygen permeability due to the porous structures. The present system monitors tear glucose in the range of about 0.02 to about 50 mmol/L. A wireless readout system converting the optical signal to the digital signal is disclosed herein and the optical signal is able to be recorded by a computer, or a cellular phone.

This present system/device can continuously detect glucose in body fluid, e.g. tears and allows a needleless and cost-efficient diagnostic testing in diabetic patients. This nanostructured contact lens-based system is a safe, sensitive, cost-effective, and non-invasive glucose monitoring solution for diabetics.

The nanostructured Luminescent Resonance Energy Transfer (LRET) sensor is made of nanostructured LRET pair-conjugated enzyme, which has highly selectivity and sensitivity for detecting glucose because enzyme as glucose recognizer is immobilized on nanoscale (1 to 10 nm).

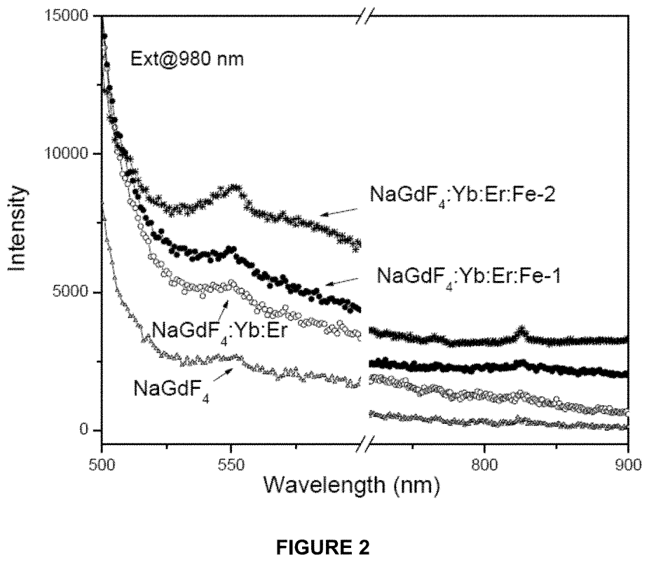

An advantage of the LRET glucose sensor is it is able to convert the bioprocess (glucose interacting with the enzyme) to a detectable fluorescent signal quickly and precisely without damaging tissues. The conjugated enzymes have strong affinity with glucose, and may include Con A, GPB, etc. There are two types of nanostructured LRET sensors. (1) NIR/IR excited LRET technique in which the donor is made of magnetic element-doped upconversion nanomaterials. (2) Bioluminescent resonance energy transfer technique in which the donor is a bioluminescent protein. Two types of donor in the can be used in the LRET glucose sensor. FIG. 2 shows the fluorescence emission of the magnetic nanostructure doped upconversion nanomaterials.

The acceptor of the LRET sensor can be made of porous fluorescent silica nanoparticles, quantum dots, and other type of nanostructures, such as silicon, and ZnO nanoparticles and nanorods, and fluorescent molecules, e.g. FITC. The nanostructured LRET sensors have high sensitivity to physiological glucose. The nanostructured LRET sensors disclosed herein have tunable fluorescent emission in the range of visible-near infrared wavelength. The nanostructured LRET sensor is assembled on contact lens with a pre-selected desired pattern to gain high sensitivity and high resolution of readable signals. Through a vapor deposition method, the sandwich-like structure is able to detect the glucose, and inhibit protein-sticking and prevent from the biofilm growth. Detection methods are flexible and feasible to conjugate a blue-tooth technical system. Bluetooth techniques can be embedded with the readout system for self-management and remote-diagnosis. Using algebra method to calibrate the detected signals, the device is able to be used for continuous measure.

The new luminescent resonance energy transfer (LRET) optical sensor is able to monitor glucose level for at least 5 days. It is noted that the matrix of the sensor is similar to the weekly wearing contact lenses. Both fluorescence intensity (1) and resonance energy transfer (RET) as the function of time and the concentration of glucose (0.01 mmol/L 10 mmol/L) have been investigated through a fluorospectrometer. The biocompatibility of the lens sensor has been studied in vitro. No toxic effect imposes on cell/tissue culture.

The most significant advantages of using up-conversion nanostructures include: (1) the nanostructures act as analyte (tear glucose,) collector to achieve high concentration of analyte reacting with the LRET enzyme sensor due to the large surface area to volume; (2) the nanostructures exhibit stable optical signals. (3) The large surface-to-volume ratio of enzyme-immobilized nanostructures can lead to higher selectivity for glucose sensing.

To monitor a broad range of glucose levels (0.01-50 mmol/L) quickly, with a high signal response, two different techniques are used to avoid high external energy for exciting the LRET sensor: (1) NIR/IR excited LRET technique in which the donor is made of magnetic element-doped upconversion nanomaterials. (2) Bioluminescent resonance energy transfer technique in which the donor is a bioluminescent protein.

Furthermore, multiple sensors and references will be produced using our near infrared (NIR) photolithography method. nanostructured self-luminescent RET sensors will be coated on hydrogel lens materials, silicone, poly(2-hydroxyethyl methacrylate) (pHEMA). To increase the application scope, both of the commercial contact lenses and lab-made hydrogel lenses will be applied to integrate the multiple enzyme-based nanostructed sensors and references with 2-D pattern. The optical transmission and oxygen permeability of the sensor holder made of hydrogels maintain standard of commercialized contact lens.

Up-conversion materials have been suggested as promising alternative fluorescent probes due to their long emission lifetimes, higher photochemical stability and low toxicity. Our findings include that (1) magnetic elements doped up-conversion nanostructures show improved emission efficiency under an NIR excitation, which can be used as a donor in LRET sensor. (2) Up-conversion nanostructures can be modified with amine function group to conjugate Enzyme which is affinity to glucose, e.g. Con A, GBP, etc. (3) Acceptor in this LRET sensor can be: nanostructrues (quantum dots, fluorescence nanostructures), fluorophores (organic dye, natural fluorescent proteins) with an excitation at 500-600 nm, and an emission in the range of 500-700 nm.

As used herein, the “upconversion nanomaterials” have the emission of light at shorter wavelength than the excitation wavelength. The magnetic nanostructrues can made of iron (Fe), nickle (Ni), cobalt (Co). The enhanced emission is able to be detected as shown in FIG. 1. Two emission peaks are observed at 510 nm, and 620 nm, respective. Compared to the upconversion nanomaterials, NaGdF₄:Yb:Er, without magnetic elements (e.g. Fe, Ni, or Co), the magnetic nanostructures-doped NaGdF₄:Yb:Er show enhanced emission as shown in FIG. 2. The results indicate the emission at both peaks (550 nm and 620 nm) improved over 80% compared to NaGdF₄ based upconversion nanostructrues, e.g. NaGdF₄:Yb:Er without Fe.

The experiment process for producing Fe-doped NaGdF₄ based up-conversion nanostructrues are described as follows:

(1) Prepare Solution A:

Gadolinium(III) nitrate hexahydrate 720 mg
Erbium(III) nitrate pentahydrate 170 mg
Ybterbium(III) nitrate pentahydrate 160 mg
Iron(III) nitrate nonahydrate (80 mg, 160 mg, 320 mg etc.)

### PEI 0.7 g

Add the above chemicals into 20 mL Ethylene Glycol and disperse by stirring at room temperature.

(2) Solution B:

Add Sodium fluoride (336 mg) into 10 mL Ethylene Glycol and sonicate to get a clear solution.

Add solution B to solution A dropwise and increase the reaction temperature to about 200° C. and refluxing for 6 hours.

The product are washed and purified by ethanol and water and centrifuge for 3 times and dried in at 60° C. to get the nanoparticles powder.

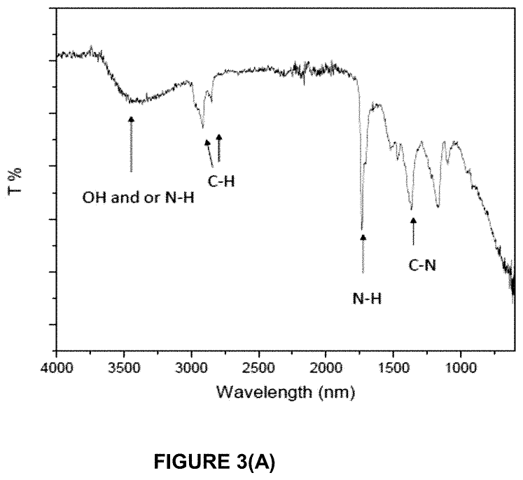

The results indicate the emission at 550 nm improved over 80% compared to NaGdF₄ based up-conversion nanostructrues, e.g. NaGdF₄:Yb:Er without Fe. The amine (—NH₂) modified on the up-conversion nanostructures was characterized by FTIR as shown in FIG. 3a. The magnetic nanostructures-doped NaGdF₄:Yb:Er are measured by transmission electron microscope (TEM). FIG. 3b indicates the average size of the magnetic nanostructures-doped NaGdF₄:Yb:Er is estimated at 35±5 nm.

A Linker is normally used to conjugate the LRET donor/acceptor (quantum dots, malachite green, fluorescence nanostructures, or gold nanoparticles) to the enzyme, which can be replaced by glucose. For instance, the conjugation of malachite green used as an acceptor/quenching element can be realized by using dextran. In short, malachite green (MG) isothiocyanate and 70,000 MW amino-dextran purchased from Life Technologies (Burlington, Ontario, Canada) were mixed in a sodium bicarbonate buffer (0.05M, pH 9.6).

Another LRET donor in this luminescent transducer is made of bioluminescent nanostructures.

The bioluminescence resonance energy transfer-fluorescence (BRET) transducer is composed of a fluorescent pair conjugated with enzyme, i.e. Con A, GBP, glucose oxidase enzyme, boronic acid. A bioluminescent protein Renilla luciferase (Rluc) is used as a donor for this BRET sensor. This recombinant protein consists of a protein, e.g. Con A, bacterial glucose binding protein (GBP), at the N-terminal and a bioluminescent protein Renilla luciferase (Rluc) at the C-terminal. Rluc could catalyze its fluorescent substrate coelenterazine (CTZ) molecules and results in emission of energy in the form of blue light with maximum wavelength at 470 nm˜500 nm. The recombinant protein was further expressed and purified from bacteria Escherichia coli BL21. Afterwards, fluorescent nanomaterials used as an acceptor can be labeled on the N-terminal of the recombinant protein. FIG. 4(a) shows the BRET sensor made of quantum dots used as an acceptor in the LRET sensor; and glucose sensitive protein-conjugated RLuc used as a donor in the sensor. Furthermore, the GBP-Rluc protein will be conjugated to the silica nanoparticles to produce the nano-switch for glucose sensing. Here, luciferase is used as a donor in BRET sensor. The experimental process is described below.

FIG. 4(b) shows the RLuc-conjugating glucose binding protein (GBP). Bacterial glucose binding protein (GBP) was cloned from E. coli k-12. Rluc gene was cloned from the plasmid pRL-null (Promega, Inc). The following primers were designed for construct the GBP-Rluc recombinant protein.

### For GBP Cloning:

(forward primer) GBPA-FP:
5′ TATACATATGAATAAGAAGGTGTTAACCCTGTCTGC 3′
(reversed primer) GBPB-RP:
5′ GCTGGATCCTTTCTTGCTGAATTCAGCCAGGTTG 3′

### For Rluc Amplification:

Linker-Rluc-FP:5′

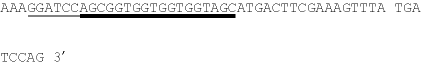

Rluc-RP : 5′ TGTGCTCGAGTTGTTCATTTTTGAGAACTCGCTC 3′

GBPA-FP and BGPB-RP introduced restriction site Nde I and Bam H I (restrict enzyme) respectively (underline). Linker-Rluc-FP and Ruc-RP introduced restriction site Bam H I and Xho I, respectively (underline). The bold underline indicates a six amino acid linker (SGGGGS) was inserted after Bam H I site to separate the sequence of GBP from that of Rluc.

The above PCR products were further digested with relating restriction enzyme. The plasmid pET 32 a (Novagen, Inc) was used to clone and express the recombinant gene. The digested DNA insert were ligated into the relating MCS (multiple cloning) site at pET 32a. A six histidine tail was introduced into the GBP-Rluc recombinant protein. FIG. 1 shows the schematic illustration of the sequence of GBP-Rluc recombinant protein. The pET 32a-GBP-Rluc was transformed into E. coli BL21 cells. The DNA sequence of the recombinant plasmid was confirmed by DNA sequencing (Robarts Institute, Western University).

The above bacterial cells with pET32 a-GBP-Rluc were grown overnight at 37° C. in 5 mL of Luria Bertani (LB) broth containing 100 μg/mL ampicillin. This culture was used to further inoculate 500 mL of broth containing 100 μg/ml ampicillin, and this was grown at 37° C. When the culture reached an OD600 of 0.375, IPTG was added to 1 mM final concentration to induce the expression of GBP-Ruc and the bacteria were left to grow for 4 hrs at room temperature. The cells were harvested by centrifugation at 12,000 rpm for 5 min at 4° C. The pellet was resuspended in a binding solution (BS) of 20 mM Tris/HCl, pH 7.4, 500 mM NaCl and 5 mM imidazole and sonicated on ice using 15-s bursts followed by 30-s rest for 30 cycles using a Mandel Scientific Q500 sonicator (Guelph, Canada).

The suspension was centrifuged at 10,000 rpm at 4° C. for 30 min to collect the supernatant from bacterial cell pellet. The protein was purified via His-trap HP columns (GE lifescience, Inc.) by a syring pump. The column was first equilibrated with BS. The supernatant containing the protein was loaded on the column, and the column was washed with 10 column volumes of the BS. The protein was eluted using BS with a gradient of imidazole from 20 mM to 200 mM) over 10 column volumes. Five milliliters fractions were collected. An SDS-PAGE was run to verify the fractions containing the fusion protein, which were pooled together. Excess imidazole was removed from the combined fractions by buffer exchange with excess amount of 10 mM Tris/HCl, pH 7.4 using an Amicon Ultra centrifugal filter (ultra-15, MWCO 10 kDa, Millipore Inc). The resultant GBP-Ruc protein solution was stored in aliquot at −20° C. The concentration of the protein was determined by Bicinchonici acid (BCA) protein assay (Thermo scientific Inc.).

The conjugation of fluorescence elements to Rluc-enzyme can use the reaction of β-cyclodextrin (β-CD) to Rluc-enzyme, or dextran to Rluc-enzyme. In brief, a dimethylformamide (DMF) solution containing 3.78 mg of succinyl-β-cyclodextrin (˜2 μmol) in 350 μL PBS was mixed with 250 μL of 10 mg/mL NHS and 400 μL of 16 mg/mL EDC. The mixture was incubated for 2 hrs at room temperature under gentle shaking. For the conjugating reaction, 200 μL of the above solution was mixed with 200 μL of 10 mg/mL Rluc solution in a PBS solution (final volume to 1 mL). The solution was further incubated overnight at 4° C. The reaction was terminated by addition of 5 μL of ethanolamine. The β-CD labeled Rluc (β-CD-Rluc) was purified through a Nap-10 column (GE Healthcare) with PBS as an eluent. The labeled protein was collected by Amicon ultral filter (ultra-15) to desired concentration and stored at 4° C. for at least four weeks without loss of more than 10% activity.

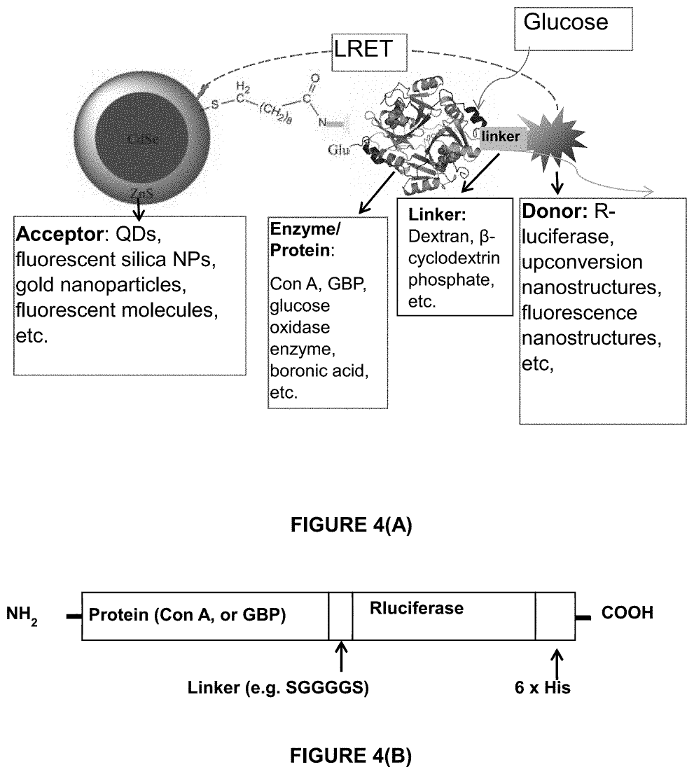

The LRET method is based on the dual-measure, i.e. the resonance energy transfer (RET) and fluorescence intensity measurements. Another novel RET donor disclosed herein can be used in this LRET transducer is applying bioluminescent resonance energy transfer (BRET)-fluorescence pair in which luminescent resonance energy transfer (LRET) is a distance-dependent energy transfer from a fluorophore donor (D) to a fluorophore acceptor (A) in a nonradiative process. In this disclosure, a BRET fluorescence conjugated with enzyme, i.e. Con A, GBP to form a BRET transducer. A bioluminescent protein Renilla luciferase (Rluc) is used as a donor for this BRET sensor. This recombinant protein consists of a protein, e.g. Con A, bacterial glucose binding protein (GBP), at the N-terminal and a bioluminescent protein Renilla luciferase (Rluc) at the C-terminal. Rluc could catalyze its fluorescent substrate coelenterazine (CTZ) molecules and results in emission of energy in the form of blue light with maximum wavelength in the range of 470 nm˜550 nm. The recombinant protein was further expressed and purified from bacteria Escherichia coli BL21. Afterwards, fluorescent nanomaterials used as an acceptor can be labeled on the N-terminal of the recombinant protein. The BRET sensor disclosed herein made of Rluc (donor) and fluorescent nanostructrues (acceptor) including fluorescent silica (SiO2), quantum dots, and fluorescent gold (Au), and a glucose sensitive protein, (Con A). Other glucose sensitive proteins are glucose binding protein (GBP). FIG. 5 shows the BRET signal of Rluc conjugated Con A which binds to CdSe-based quantum dots. The BRET signal response 0.04 mM glucose is shown in FIG. 5 as well.

FIG. 5 shows a spectra of the BRET sensor and the BRET signal responding to 0.04 mM glucose. To improve the detection accuracy in a convenient manner, the LRET transducer is integrated on a contact lens made of silicone.

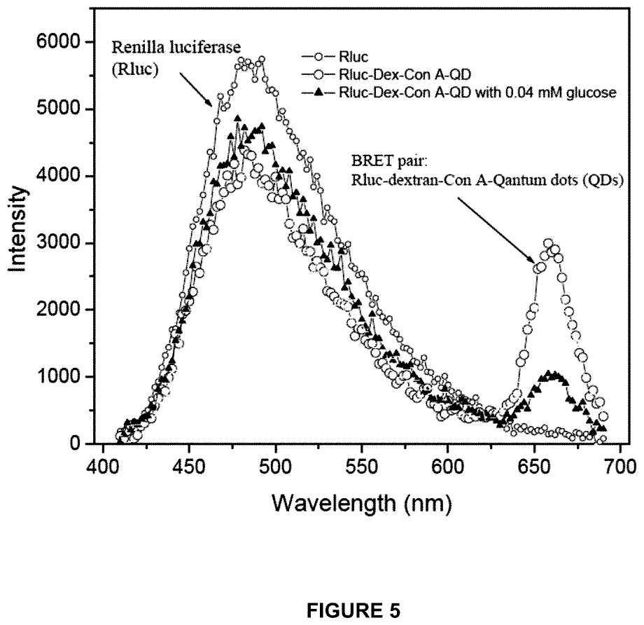

FIG. 6a illustrates a LRET transducer made of hybrid nanostructures coated on a hydrogel substrate. The amplified fluorescence signal will be achieved due to this design because multiple nanostructured LRET sensors can be assembled on one nanorod which is directly deposited or grown on the hydrogel substrate. The hybrid nanostructures herein include (1) a type of nanostructures directly deposited/grown on a hydrogel substrate, which further act as a supporter to be assembled as one or multiple LRET transducers. This type of nanostructures directly deposited on hydrogel substrates can be nanorods, nanobelts, nanotubes, and nanoparticles. This type of nanostructures can also act as either a donor or an acceptor of the assembled LRET sensors; (2) assembled one or multiple nanostructured LRET sensors. One LRET sensor includes the donor of LRET sensor, the conjugated enzyme which can interact with glucose, an acceptor of LRET sensor, and linkers to bind the donor, the enzyme, and the acceptor.

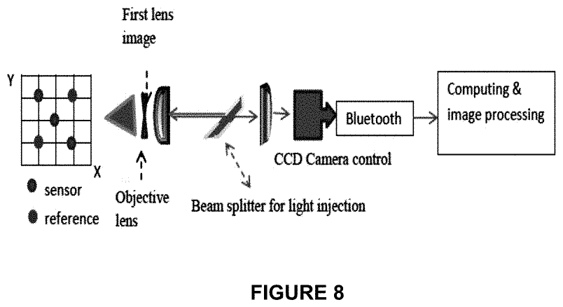

FIG. 6b illustrates the patterned nanostructured LRET sensor coated on a silicone hydrogel substrate. This design enables calibrating of the image pixel intensity. There are three areas: (1) positive control which generates the highest LRET signal, (2) designed patterns by depositing nanostructured LRET sensors on a transparent substrate for detecting glucose, and (3) negative control which exhibits the lowest LRET signal. The detailed information on the calibrated image pixel intensity value is described in FIG. 9.

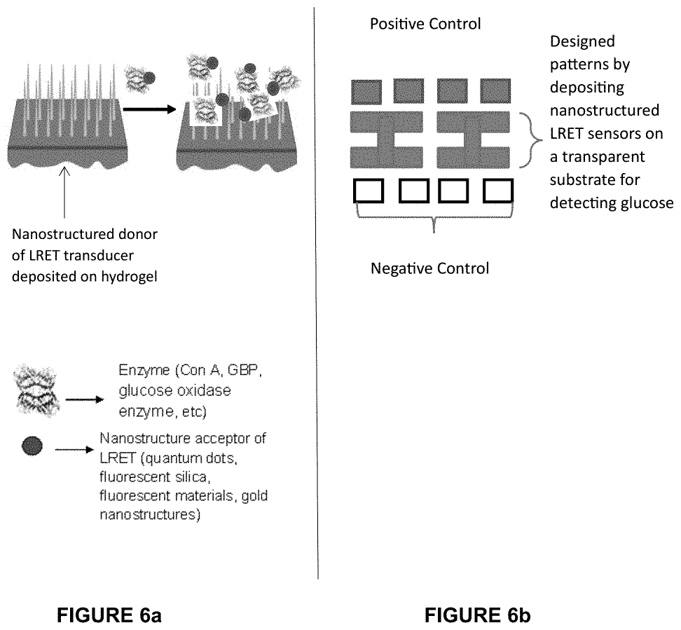

The patterned LRET sensors assembled on a silicone substrate was measured by a scanning electron microscope as shown in FIG. 7. The pattered LRET assembled on the lens materials (silicone, pHEMA) allows the detection of LRET signals corresponding to the concentration of glucose in an accurate fashion.

Therefore, a developed readout system enables one to detect the patterned LRET sensors for helping patients to have a convenient and accurate disease management. The present system may employ a real-time algorithm and calibration to minimize the effects of time lag, various background lights, and to amplify the detected signals using the system shown in FIG. 8.

The detected fluorescence spectra and captured images generated from three major spots with the same areas of the device as shown in FIG. 8. That is, one sensing area, and two reference areas. The sensing area provides the fluorescence signal from the LRET sensor embedded in the transparent substrate, including hydrogel-based contact lenses, glass, polydimethylsiloxane. The signal from this area depends on the concentration of glucose. There are two reference areas on the device which do not react with glucose. One reference area, which has no LRET sensor, acts as a negative control, and therefore provides the signal of the substrate or the lowest fluorescence signal. The other reference area acts as the positive control, which is only the part of the light emitting donor of the LRET sensor, and provides the highest luminescence signal. Once the LRET sensor interacts with body fluids which contain glucose, the detected fluorescence spectra or captured fluorescence images can be further processed through an algebra method executed on a computer processor in comparison with the negative control and positive control. The detailed algebra method is discussed as follows. At a certain of concentration of aqueous glucose [C], the corresponding optical signal for the nanostructured LRET sensors encoded on lens materials, e.g. intensity (I), and/or wavelength (λ), will be processed based on Equation-1 as below;

$${I}_{R} = \frac{I - {I}_{negative}}{{I}_{positive} - {I}_{negative}} \qquad Equation \text{-} 1$$

Where I_{R} is the calculated intensity of the LRET sensor after the calibration. I is _{fluorescence} intensity of the sensing area corresponding to the amount of glucose, I_{negative} the fluorescence intensity of the negative control area, Iₚₒₛᵢₜᵢᵥₑ the fluorescence intensity of the positive control area. The fluorescence signal reading corresponding to the glucose concentration will be more accurate by locating the detection areas. Such efforts on noise mitigation will improve the resolution and sensitivity of the designed glucose sensing system.

Integrating nanostructured lens-based glucose sensor with a wireless transmitter will enable efficient control the insulin release. Meanwhile, the detection results can be further shared by the patient with his/her doctors enabled by the communications capabilities of the smartphone, including using social networks such as Twitter and Facebook to eventually build an accurate, continuous and remote monitoring system for diabetics who need to have regular insulin treatment.

FIG. 9 shows data on converting the image of lens sensor in different concentrations of aqueous glucose to the readable signal. There are several available techniques to obtain eye imaging in vivo. Here, the proposed system is adapting the technique of optical coherence tomography (OCT) to capture the image using a charge-coupled device (CCD) on the surface of lens, and convert the image to digital signal based on the relationship of the imaging (1) vs. the concentration of glucose (C). Patterned coating multiple nanostructured LRET sensors on lens materials, through spatially encoded patterns, will be constructively combined to mitigate the noise in the detection image. The LRET glucose sensors and two reference areas which do not react with glucose are embedded in the transparent substrate. The fluorescence images of the three areas are captured as shown in FIG. 9. The negative control refers the image of the substrate or the area with the lowest fluorescence signal, the positive control refers the captured image of the area with the light emitting donor. The captured fluorescence image of the LRET sensing area that interacts with body fluids which contains glucose can be further processed in comparison with the negative control and positive control. FIG. 9 shows two fluorescence images of patterned LRET sensor interacting with different concentration of glucose, and the corresponding values of the pixel intensity by using the MatLab imaging process. Sample A is the aqueous glucose with concentration of 0.04 mM, and Sample B is the aqueous glucose with concentration of 0.4 mM. The recorded images by the fluorescence camera were converted to the readable signal through Matlab's imaging process. The captured LRET sensing image with pixels ΣXY is calibrated in comparison with the image the embedded two reference areas: negative control area with pixels ΣX′Y′ and positive control area with pixels ΣX″Y″ The calibatrated pixel intensity (Iₚ) generated from LRET sensors can be expressed as follow;

Iₚ=[Σ^{I}(XᵢYᵢ)−Σ^{I}(Xᵢ′Yᵢ′)/Σ^{I}(Xᵢ″Yᵢ″)−Σ^{I}(Xᵢ′Yᵢ′)], Equation-2

where, I is the pixel intensity, X and Y are the position of the LRET sensor, X′ and Y′ are the position of the negative control, and X″ and Y″ are the position of the negative control. The algebra method can be applied in obtaining the relative pixels of LRET sensors corresponding to the amount of glucose. As a result, CCD optical detector connecting a Bluetooth device will transmit the image with glucose level induced color changes to a smartphones for accurate, real-time, and continuously measure.

Silicone is a good candidate as a hydrogel material due to its composition of siloxane groups which can carry large amounts of oxygen. This new transport mechanism results in higher oxygen transmissibility than conventional hydrogels. In addition, silicone's good biocompatibility, transparency, stable chemical structure and proper mechanical strength make it suitable for biomedical applications. However, due to its hydrophobic surface, silicone adsorbs protein easily. Polyethylene glycol (PEG), as a surface coating, has been shown to decrease protein adsorption due to its hydrophilic properties and extremely low toxicity.

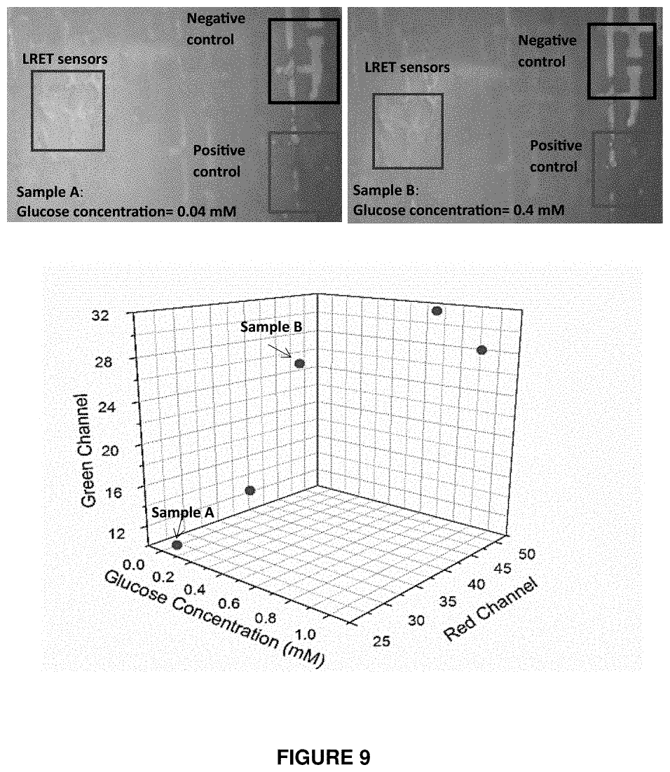

In this disclosure, a hydrophilic polymer (PEG) coating is deposited on the contact lens based LRET sensor as shown in FIG. 10 through a matrix-assisted pulsed laser evaporation process. This coating can enhance the biocompatibility and inhibit the growth of biofilm. To ensure retention of the biological function of deposited organic molecules. A 5% solution (or less) of the material is prepared and frozen with liquid nitrogen. The energy of the laser is mostly absorbed by the solvent to reduce the damage to the target molecules. Excimer lasers or Nd: YAG lasers with the third harmonic at 335 nm are the laser sources mostly suited for MAPLE; infrared laser sources are utilized in particular cases.

Protein adsorption on artificial implants may cause an inflammatory response in the human body, therefore the protein adsorption of hydrogels were tested. The samples were immersed in distilled water overnight, and then soaked in 0.5 mg/ml bovine serum albumin (BSA)-PBS solution for 3 h at 37° C. After that, the samples were rinsed with PBS solution thrice to remove non-adsorbed BSA. The samples were then immersed in a 1% wt SDS-PBS solution and sonicated for 20 min to completely detach BSA from the hydrogel surface. Finally, the BCA protein assay kit (Micro BCA™ Protein Assay Kit, Thermo Scientific, USA) was used to determine the protein concentration in the SDS-PBS solution with a UV-visible plate reader at a wavelength of 562 nm.

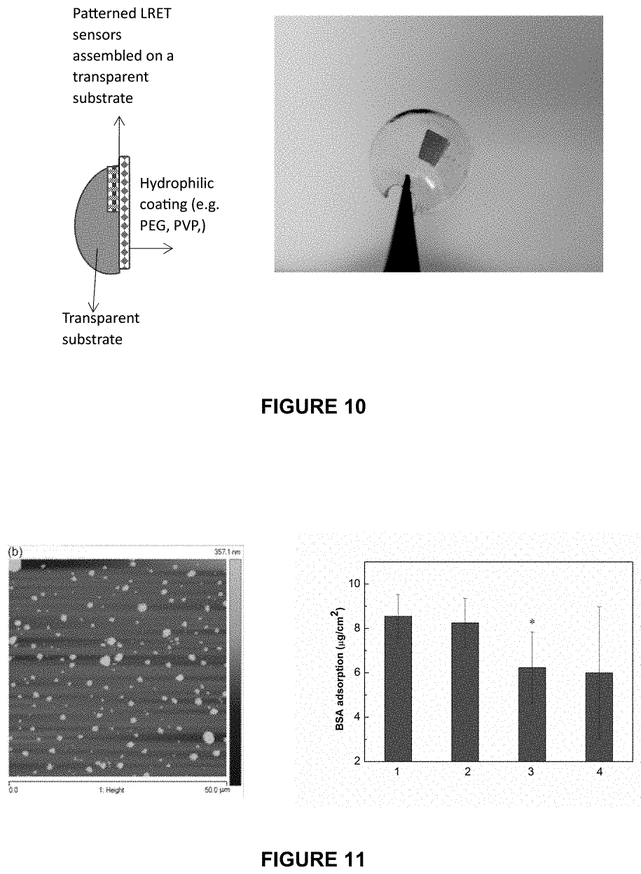

FIG. 11 shows BSA adsorption of silicone and its nanocomposite with/without PEG deposition by MAPLE. *Significant difference was found between 1 and 3 (p<0.05). 1—silicone; 2—silicone-SNPs; 3—PEG deposited on silicone; 4—PEG deposited on silicone-SNPs

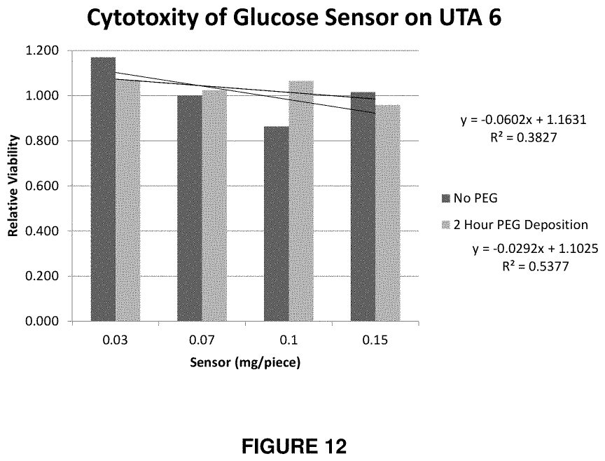

Meanwhile, the cytotoxicity of the LRET sensor embedded in hydrogel-based contact lens have been studied. 50,000 3T3 mouse fibroblast cells were seeded into a 24 well culture plate and incubated in a 5% CO2 incubator overnight. Hydrogels and nanocomposites were cut into 0.5 g pieces and incubated with cells for 24 hours. Cell viability was accessed by using a 3-(4,5-Dimethylthiazol-2-yl)-2,5-diphenyltetrazolium bromide (MTT) Assay. Briefly, after remove the samples, the MTT reagent was added to 24-well plate and incubated at 37° C. for another 4 h, then DMSO was added to dissolve the purple formazan product. The resulting signals were measured at an absorbance of 490 nm. FIG. 12 indicates the PEG coated nanostructured LRET sensor assembled on hydrogels do not impose any toxic effect on cells.

Furthermore, multiple sensors and references will be produced using our near infrared (NIR) photolithography method. The nanostructured self-luminescent RET sensors may be coated on hydrogel lens materials, silicone, poly(2-hydroxyethyl methacrylate) (pHEMA). To increase the application scope, both of the commercial contact lenses and lab-made hydrogel lenses will be applied to integrate the multiple enzyme-based nanostructured sensors and references with 2-D pattern. The optical transmission and oxygen permeability of the sensor holder made of hydrogels maintain standard of commercialized contact lens.

While the sensor described above has been with reference to its use in conjunction with a hydrogel based contact lens for detecting glucose in tears, it will be appreciated this is only an exemplary embodiment. Any other body fluid may be tested, including urine, saliva and blood to list some non-limiting examples. For example, when the fluid is urine the transparent substrate is any one or combination of a hydrogel-based materials, polyurethanes, glass and polydimethylsiloxane. When the fluid is saliva the transparent substrate may be any one or combination of hydrogel-based materials, polyurethane and polydimethylsiloxane to give a couple of non-limiting examples.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

### REFERENCES

[1] http://advocacy.jdrf.org/files/General_Files/General_Files/AP_Fact_sheet.pdf
[2] JDRF randomized clinical trial to assess the efficacy of real-time continuous glucose monitoring in the management of type 1 diabetes: research design and methods. Diabetes Technol Ther. 2008; 10(4):310-21.
[3] Deiss D, Bolinder J, Riveline J P, et al. Diabetes Care. 2006; 29(12):2730-2.
[4] Tamborlane W V, Beck R W, Bode B W, et al. N Engl J Med. 2008; 359(14):1464-76.
[5] Gough D A, Kumosa L S, Routh T L, Lin J T, Lucisano J Y. Sci. Transl. Med. 2010; 2:42-53.
[6] Onuki Y, Bhardwaj U, Papadimitrakopoulos F, and Burgess D J, J Diabetes Sci Technol. 2008; 2(6): 1003-1015.
[7] Ohahi Y, Dogru M, and Tsubota K, Clinica Chimica Acta. 2006; 369:17-28.
[8] An H J, Ninonuevo M, Aguilan J, Liu H, Lebrilla C B, Alvarenga L S, and Mannis M J, J. Proteome Res. 2005; 4: 1981-1987.
[9] Ridley F, Br J Exp Pathol. 1930; 11:217-220.
[10] Michail D, Vancea P, Zolog N. C R Soc Biol (Paris) 1937; 125:1095-1099.
[11] Sen D K and Sarin G S, Bri. J. Ophthalmol. 1980; 64: 693-695.
[12] Lane J D, Krumholz D M, Sack R A, Morris C. Curr Eye Res. 2006; 31:895-901.
[1] Gasset A R, Braverman L E, Fleming M C, Arky R A, Alter B R. Am J Ophthalmol. 1968; 65: 414-420.
[14] Motoji K. Jpn J Clin Ophthalmol 1971; 25: 1945-1950.
[15] Daum K M, Hill R M. Investigative Ophthalmology & Visual Science. 1982; 22:509-514.
[16] Lewis J G, Stephens P J. Br J Ophthalmol 1958; 42: 754-8.
[17] Bjerrum K B and Prause J U, Graefe's Arch Clin Exp Ophthalmol. 1994; 232: 402-405.
[18] Martin D and Fatt I, Acta Ophthalmologica. 1986; 64: 512-518.
[19] O'Donnell C, Efron N, Boulton A J. Ophthal Physiol Opt 2001; 21:127-138.
[20] March W, Long B, Hofmann W. Diabetes Technol Ther 2004; 6:49-52
[21] Alexeev V L, Das S, Finegold D N, Asher S A. Clin Chem 2004; 50:2353-2360
[22] Ramachandram Badugu, 1 Joseph R. Lakowicz, 1,3 and Chris D. Geddes1, Journal of Fluorescence, 2004; 14: 617-633
[23] Bhupinder Singh Sekhon, Current Chemical Biology, 2008; 2:278-311.
[24] http://www.bd.com/us/diabetes/page.aspx?cat=7001&id=32426
[25] Peter H. Kvist, and Henrik E. Jensen, Journal of Diabetes Science and Technology, 1, Issue 5, September 2007 746-752.
[26] Zhang J and Hodge W, U.S. patent Ser. No. 12/588,733; Canadian patent No. 886171024.
[27] Zhang J, Hodge W, Hutnick C, and Wang X, J. Diabetes Sci. Tech. 2011; 5:166-170.
[28] Auzel F., Chem. Rev., 2004, 104 (1):139-174

1-25. (canceled)

26. An apparatus for the detection of glucose levels in body fluids, comprising
the nanostructured arrays extending upwardly from a surface which displays repeated design and can be recognized by a camera or a CCD camera; and
a plurality of luminescent sensors immobilized on the nanostructured arrays can produce a detectable visible emission once interacting with glucose molecules without shining visible or UV light. wherein each of said luminescent sensors comprises
a light energy donor wherein the donor is bioluminescence compounds, or upconversion structures.
alight absorbing and/or emitting acceptor, and
a glucose recognition agent, wherein the glucose recognition agent is placed in the middle of the light donor and the light absorbing and/or emitting acceptor through linker molecules
in response to interaction with glucose contained in a body fluid, the restore or increase of bioluminescence or upconversion signals is a function of the concentration of glucose interacting with the glucose recognition agent.

27. The apparatus of claim 26 wherein said nanostructured arrays extending upwardly from a surfaces are the form of nanorods, nanobelts, nanotubes, or nanoparticles.

28. The apparatus of claim 26 wherein said nanostructured arrays extending upwardly from a surfaces are modified with functional compounds including, hydroxyl, ester, aldehyde, carboxylic acid, acid halide, amide, amine, silicones, siloxanes and silsesquioxanes.

29. The apparatus of claim 26 wherein said nanostructured arrays extending upwardly from a surface are produced by nanolithography, photolithography, atomic force-based lithography.

30. The light energy donor of claim 26 is made of a bioluminescence compound, which is made of protein-based or recombinant protein-based compounds containing the sequences of bacterial luciferase, firefly luciferase, Renilla luciferase.

31. The light energy donor of claim 26 is made of an upconversion structures, which is any one or a combination of rare earth-based nanomaterials, and magnetic element-doped upconversion nanomaterials.

32. The apparatus of claim 26, wherein the light absorbing and/or light emitting acceptor includes any one or combination of porous fluorescent silica nanoparticles, quantum dots, silicon, ZnO nanoparticles, nanorods, metallic nanoparticles and fluorescent nanostructures.

33. The linker molecule of claim 26 is any one or combination of dextran, 3-cyclodextrin, phosphate, a nucleotide-based linker, and an amino acid-based linker.

34. The apparatus of claim 26, wherein the glucose recognition agent has affinity with glucose, including any one or combination of protein-based molecules, enzyme-based molecules, and synthetic chemical molecule, including any one or combination of glucose binding protein (GBP), Concanavalin A (Con A), glucose oxidase enzyme, and boronic acid

35. The restored or increased signals come from the luminescent sensor after interacting with glucose according to claim 26 can be analyzed by a detection system.

36. The detection system of claim 35, wherein the signal detector is a fluorescence camera configured to detect fluorescence images and/or provide a fluorescence spectral response.

37. The apparatus according to claim 26 wherein said body fluid is tears, and wherein said the surface is that of a hydrogel-based composites.

38. The apparatus according to claim 26 wherein said body fluid is urine, and wherein said the surface is any one of a hydrogel-based composites, polyurethane-based composites, glass and polydimethylsiloxane-based composites.

39. The apparatus according to claim 26 wherein said body fluid is saliva, and wherein said the surface is any one of a hydrogel-based composites, polyurethane-based composites, glass and polydimethylsiloxane-based composites.

40. The recombinant protein of claim 30 wherein said can emit luminescence without fluorescence excitation, wherein the recombinant protein can be expressed in bacteria, e.g. Escherichia coli.

41. The detector system of claim 35, for use in association with a computer processor configured for calibrating a detected luminescent signal generated from the plurality of luminescent sensors integrated with the nanostructured arrays through calculating the effects of environmental conditions.

42. The detector system of claim 35, for use in association with a computer processor configured for converting the luminescent pixel intensities by an algorithm method executed on the computer processor as a function of glucose concentration.


SEQUENCE LISTING (3 sequences)

SEQ ID NO: 1 - RNA, 698 nt, Cyphonocerus ruficollis
  ref <301>: AUTHORS: Oba Y., Yoshida M., Shintani T., Furuhashi M., Inouye S.
  ref <302>: TITLE: Firefly luciferase genes from the subfamilies Psilocladinae and Ototretinae (Lampyridae, Coleoptera)
  ref <303>: JOURNAL: Comp. Biochem. Physiol. B, Biochem. Mol. Biol
  ref <304>: VOLUME: 161
  ref <305>: ISSUE: 2
  ref <306>: PAGES: 110-116
  ref <307>: DATE: 2012-02-15
aggagaggaagaaaaaagggacgggccagcgccaacccgggagaggaacagcagggaacaacccacaaaacgaaagaagcccaacacggaacacgcaacggagcacaacgaagaaacgacaagcagagaagagcaacagcgcggcagaagccagaaaaggaggacgaaaaacaagaagcagagaggaaaaagcggagcggccagaagcgccaaccggggagccgagcccaacaaagacaacaagaacgggaagaaagaaaaacgcaaccacaagcaccaaaaagagcacaaaaaggaaggcaaaagcaaaccggaaagaaaacgagaagaagcaaagagacagggaagcaacaagcaccacacaagcacaccaccagcagaaacgaacagaaaaaccgaaagcgacgaaagaacaaggcgcaaagaacacggacaacaggaaccaaaaggaggcaaaacgcaaaaaagagagcagacccacgcaaagaccgaggaacaaaaccgaacgcaaacgaaccacacaggggagacaacaaggaagcacgggacgagggaagcacaaagaggaagaacaacgaccccaagaacaaaaccaaaggcaacggaccgacagcgcgcaaaagacacgggaaaaagaacaaaacacgaaagccggggagcacccagcaaaggaagcggggaggcggcaaaacggaaac

SEQ ID NO: 2 - protein, 1000 aa, Cyphonocerus ruficollis
  ref <301>: AUTHORS: Oba Y., Yoshida M., Shintani T., Furuhashi M., Inouye S.
  ref <302>: TITLE: Firefly luciferase genes from the subfamilies Psilocladinae and Ototretinae (Lampyridae, Coleoptera)
  ref <303>: JOURNAL: Comp. Biochem. Physiol. B, Biochem. Mol. Biol.
  ref <304>: VOLUME: 161
  ref <305>: ISSUE: 2
  ref <306>: PAGES: 110-116
  ref <307>: DATE: 2012-02-15
ATGGAGATGGAAGATAAAAATGTGGTACGTGGTCCAGCGCCATTTTACCCTGTGGATGATGGAACAGCAGGTGAACAACTCCACAAAACGATTTTAAGATATGCCCAACTATCTGGAACTATCGCATTTACGGATGCACATACTGAAGTAAACGTTACATATGCAGAGTATTATGATGCAACATGTCGCTTGGCAGAAGCCATGAAAAGGTATGGACTTGATTTAAAACATAGAATTGCAGTATGTAGTGAAAATAGTCTGGAGTTCTTTGTGCCAGTAATTGCTGCTCTATACCTTGGGGTAGCCGTAGCTCCAACAAATGACATTTACAATGAACGGGAATTGTTTAATAGTTTAAATATATCGCAACCTACTATAGTTTTCACTTCAAAAAGAGCTTTACATAAAATTTTGGAAGTGCAAAAGCAATTACCGGTTATTAAGAAAATCGTAGTATTAGATAGCAAAGATGACTTTATGGGAATGCAATCAATGCACTCATTCATCAAGCACTACCTACCAGCAGATTTTAACGAACTAGATTATAAACCTGAAAGCTTTGATCGAAATGAACAAGTTGCGCTTATAATGAATTCATCTGGATCAACAGGATTACCAAAAGGAGTGCAATTAACGCATAAAAATGTAGTAGTCAGATTTTCCCACTGCAAAGATCCTGTATTTGGTAATCAAATTATTCCTGATACTGCTATTTTAACTGTTATACCATTTCATCATGGTTTTGGTATGTTTACAACATTAGGATATGTCACTTGTGGATTTCGTATTGTGGTAATGCACAAATTTGAGGAAGAACTATTTTTACGATCCCTTCAAGATTACAAAATCCAAAGTGCATTACTGGTACCGACATTGTTTTCGTTCTTTGCTAAAAGTACACTTGTGGATAAATATGATTTATCAAATTTACACGAAATTGCTTCTGGTGGAGCACCTCTAGCAAAGGAAGTCGGGGAGGCTGTTGCAAAACGGTTTAAAC

SEQ ID NO: 3 - protein, 1575 aa, Glycine max
  ref <301>: AUTHORS: Grimes HD., Overvoorde PJ., Ripp K., Franceschi VR., Hitz WD
  ref <302>: TITLE: A 62-kD sucrose binding protein is expressed and localized in tissues actively engaged in sucrose transport
  ref <303>: JOURNAL: Plant Cell
  ref <304>: VOLUME: 4
  ref <305>: ISSUE: 12
  ref <306>: PAGES: 1561-1574
  ref <307>: DATE: 1992-12-08
ATGGGCATGAGAACCAAACTTTCTTTAGCTATCTTTTTCTTTTTTCTTTTAGCCTTATTTTCAAACCTAGCCTTTGGCAAATGTAAAGAAACCGAGGTAGAAGAAGAAGACCCTGAGCTCGTAACTTGCAAACACCAATGCCAACAGCAACAGCAATACACTGAGGGTGACAAGCGGGTATGCTTGCAAAGTTGTGACAGGTATCATCGTATGAAGCAAGAGCGAGAGAAACAAATCCAAGAGGAAACTCGTGAGAAGAAAGAGGAGGAAAGCCGTGAAAGGGAAGAAGAACAACAAGAGCAGCATGAGGAACAAGATGAAAATCCCTACATTTTTGAGGAAGATAAGGATTTTGAGACCAGAGTCGAAACTGAAGGTGGCAGAATTCGGGTTCTCAAGAAGTTCACTGAAAAATCCAAGCTTCTTCAGGGCATTGAGAACTTCCGTTTGGCCATTTTGGAAGCTAGAGCACACACGTTCGTGTCTCCTCGCCACTTTGATTCCGAGGTTGTTTTTTTCAACATCAAGGGGCGAGCCGTACTTGGGTTGGTGAGTGAAAGTGAAACAGAAAAAATCACCCTTGAACCTGGGGACATGATACACATACCAGCTGGCACCCCACTGTACATCGTTAACAGAGATGAGAATGATAAGCTCTTCCTTGCCATGCTCCATATACCTGTCTCTGTCTCTACTCCTGGAAAATTTGAGGAATTTTTCGCGCCTGGAGGACGAGACCCAGAATCGGTCCTCTCAGCATTCAGCTGGAATGTGCTGCAAGCTGCGCTCCAAACCCCAAAAGGAAAGTTAGAAAACGTTTTTGATCAACAGAACGAGGGAAGTATTTTCCGAATAAGCAGAGAACAGGTGCGTGCGTTGGCCCCTACCAAGAAAAGCTCTTGGTGGCCATTCGGTGGCGAATCCAAGCCTCAATTCAATATTTTCAGCAAGCGTCCCACTATCTCCAACGGATATGGTCGTTTAACTGAAGTTGGTCCTGACGATGATGAAAAGAGTTGGCTTCAAAGACTCAATCTCATGCTTACCTTTACCAACATCACCCAGAGATCTATGAGTACTATTCACTACAACTCACATGCAACGAAGATAGCACTGGTGATCGATGGCAGAGGGCATCTTCAAATATCATGTCCACACATGTCATCAAGGTCGTCACACTCGAAGCATGATAAGAGTAGCCCTTCATACCATAGAATCAGTTCCGATTTGAAGCCTGGAATGGTGTTTGTTGTCCCTCCCGGTCATCCCTTCGTCACCATAGCTTCCAATAAAGAGAATCTCCTCATGATTTGCTTCGAGGTTAACGCTCGAGACAACAAGAAGTTTACATTTGCAGGGAAGGACAACATTGTGAGTTCTCTGGACAACGTTGCTAAGGAGCTTGCCTTTAACTATCCTTCTGAGATGGTGAACGGAGTATTCCTGTTGCAACGATTCCTCGAACGGAAATTGATAGGAAGACTCTACCACTTGCCTCATAAGGACCGAAAGGAGAGTTTCTTTTTCCCTTTTGAGTTGCCGAGAGAGGAGCGTGGTCGTCGCGCTGATGCGTGA